(19) United States
(12) Patent Application Publication
(10) Pub. No.: US 2025/0230480 A1
(43) Pub. Date: Jul. 17, 2025

(54) RECOMBINANT VECTOR AND USES THEREOF

(71) Applicant: MELLO BIOTECH TAIWAN CO., LTD., Taipei City (TW)

(72) Inventor: Hsien-Lin LIU, Taoyuan City (TW)

(21) Appl. No.: 19/017,857

(22) Filed: Jan. 13, 2025

Related U.S. Application Data
(60) Provisional application No. 63/620,959, filed on Jan. 15, 2024.

(51) Int. Cl. C12P 19/34; C12N 9/12

(52) U.S. Cl. CPC C12P 19/34; C12N 9/127; C12Y 207/07048

(57) ABSTRACT

Disclosed herein is a recombinant vector for expressing a replicase comprising a novel polydeoxyribonucleotide. The thus-expressed replicase can be used in in vitro RNA amplification via an RNA-dependent RNA cycling reaction (RCR). Also disclosed herein is a method for producing an amplified RNA product in an RNA cycling reaction (RCR) via use of the present replicase encoded by the present polydeoxyribonucleotide.

### CROSS-REFERENCES TO RELATED APPLICATIONS

This application relates to and claims the benefits of U.S. Provisional Application No. 63/620,959 filed Jan. 15, 2023; the content of the application is incorporated herein by reference in the entirety.

### SEQUENCE LISTING XML

The present application is being filed along with a Sequence Listing XML in electronic format. The Sequence Listing XML is provided as an XML file entitled P4335-US_SEQ, created Dec. 16, 2024, which is 20 Kb in size. The information in the electronic format of the Sequence Listing XML is incorporated herein by reference in its entirety.

### BACKGROUND OF THE INVENTION

### 1. Field of the Invention

The present disclosure relates to the field of nucleic acid amplification technology. More particularly, the disclosed invention relates to a recombinant vector comprising a novel polydeoxyribonucleotide, which is optimized using human codons to enhance the expression of a replicase suitable for use in an RNA-dependent RNA cycling reaction (RCR).

### 2. Description of Related Art

The RNA-dependent RNA cycling reaction (RCR) is an innovative methodology for producing an amplified RNA product in vitro directly from a designated RNA sequence found on a sense-strand RNA template. The RCR process relies mainly on the participation of RNA-dependent RNA polymerase (RdRp) for replication. During the amplification process, a sense-strand RNA template, containing at least one RdRp-binding site, is utilized alongside RNA replicases capable of recognizing the RdRp-binding site, so as to produce a complementary, antisense-strand RNA sequence that contains the original RNA template and the RdRp-binding site as well. Thus, the antisense-strand RNA sequence can serve as another template for amplifying the sense-strand RNA sequences, which further serves as templates for amplifying antisense-strand RNA sequences again. Accordingly, this iterative process enables the repeated and alternating amplification of sense-strand and antisense-strand RNAs through the RNA-dependent RCR technique, thereby efficiently producing the desired RNA.

To facilitate the above-mentioned process, the choice of replicases is crucial. However, obtaining replicases from viruses remains a challenge in the related field. Further, the viral replicases do not exhibit satisfactory amplification results.

In view of the foregoing, there exists in the related art a need of an improved replicase capable of recognizing the RdRp-binding site in an RCR, thereby achieving amplification of RNA in vitro.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a recombinant vector for expressing a replicase comprising a polydeoxyribonucleotide (PDRN) of SEQ ID NOs: 1, 2 or 3.

According to some embodiments of the present disclosure, the polydeoxyribonucleotide of SEQ ID NOs: 1, 2 or 3 is independently transcribed to a polyribonucleotide sequence of SEQ ID NOs: 4, 5, or 6.

According to some embodiments of the present disclosure, the replicase comprises an amino acid sequence of SEQ ID NOs: 7, 8 or 9.

Another aspect of the present disclosure is directed to a method for producing an amplified RNA product in an RNA cycling reaction (RCR) comprising amplifying an RNA template in the presence of a replicase expressed by the afore-mentioned recombinant vector.

According to some embodiments of the present disclosure, the RNA template comprising a polyribonucleotide sequence of a coding RNA or a non-coding RNA.

According to one embodiment of the present disclosure, the coding RNA may be a messenger RNA (mRNA) that encodes an antigen. Specifically, the antigen may be a cancer antigen, a tumor antigen, a bacterial antigen, a viral antigen, a fungal antigen, a parasitic antigen, or a combination thereof.

Example of the tumor antigen includes, but is not limited to, a neoantigen, a tumor-derived lysate, an alpha-fetoprotein (AFP), a carcinoembryonic antigen (CEA), a mucin protein, an epithelial tumor antigen (ETA), a tyrosinase, a melanoma-associated antigen (MAGE), a RAS protein, and a tumor suppressor protein.

The exemplary bacterial antigen may be derived from a bacterial species including Actinomyces, Aeromonas, Arthrobacter, Bacillus, Bacteroides, Bordetella, Borrelia, Brucella, Citrobacter, Clostridium, Corynebacterium, Escherichia, Campylobacter, Chlamydia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia; but is not limited thereto.

The exemplary viral antigen may be derived from a viral species that includes, but is not limited to, Adenovirus, Alphacoronavirus, Betacoronavirus, Cytomegalovirus, Deltainfluenzavirus, Deltacoronavirus, Gammacoronavirus, Hepacivirus, Hepatovirus, Influenza A virus, Influenza B virus, Influenza C virus, Influenza D virus, Lentivirus, Letovirus, Lymphocryptovirus, Orthopneumovirus, Orthopoxvirus, Papillomavirus, Quaranjavirus, Rotavirus, Simplexvirus, and Varicellovirus. In one preferred embodiment of the present disclosure, the viral antigen is derived from a spike protein of Betacoronavirus.

The exemplary fungal antigen may be derived from a fungal species that causes a fungal infection, which includes aspergillosis, blastomycosis, candidiasis, chromoblastomycosis, cryptococcosis, histoplasmosis, mycetoma, paracoccidioidomycosis, ringworm and tinea versicolor; yet is not limited thereto.

The exemplary parasitic antigen may be derived from a parasite species that causes a parasitic infection, which includes but is not limited to, African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, and trichuriasis.

According to some embodiments of the present disclosure, the non-coding RNA may be a small interfering RNA (siRNA), a ribosomal RNA (rRNA), a transfer RNA (tRNA), a microRNA (miRNA), or an aptamer.

According to some embodiments of the present disclosure, the RNA template is a chimeric DNA/RNA oligonucleotide.

Many of the attendant features and advantages of the present disclosure will become better understood with reference to the following detailed description.

### DESCRIPTION

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skilled in the art to which this invention belongs.

The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

The term “replicase” as used herein refers to an enzyme found in or derived from viruses that are responsible for replicating genetic materials, such as DNA and RNA, therefore plays a crucial role in copying and synthesizing genetic material during viral replication. Replicase is highly specialized for its specific nucleic acid (RNA or DNA) and maybe termed differently depending on the type of genetic material. According to embodiments of the present disclosure, examples of the replicase includes RNA-dependent RNA polymerase (RdRp), reverse transcriptase, DNA polymerases, and RNA-dependent DNA polymerases, but are not limited hereto.

The term “recombinant vector” as used herein refers to, a vector derived from a plasmid or a virus for the transportation and/or transfer of designated genes or genetic materials into a host (e.g., cells). Typically, the recombinant vector includes a DNA fragment and various functional components (e.g., origin of replication (ori) and promoter) specifically designed for expressing the DNA fragment within cells or in cell-free condition.

The term “chimeric DNA/RNA oligonucleotide” as used herein refers to an oligonucleotide consisting of RNA and DNA sequences. Typically, chimeric RNA/DNA oligonucleotides are chemically synthesized via the use of both RNA and DNA monomers. According to embodiments of the present disclosure, a nucleotide template used in RCR for RNA amplification may consist of sequences of both polydeoxyribonucleotides and polyribonucleotides. Therefore, the nucleotide template in the present disclosure is a chimeric DNA/RNA oligonucleotide.

### 2. Description of the Invention

The present disclosure is based, at least in part, on the discovery of a novel polydeoxyribonucleotide (PDRN) that encodes a replicase capable of recognizing a ribonucleic acid (RNA) template to initiate in situ transcription. Accordingly, the present disclosure provides a recombinant vector comprising a novel polydeoxyribonucleotide (i.e., SEQ ID NOs: 1, 2 or 3) for expressing a replicase, and the thus-produced replicase can be used in an RNA-dependent RNA cycling reaction (RCR) for RNA amplification in vitro. Note that the recombinant vector has been optimized using human codons to enhance the expression of the recombinant protein. Also disclosed herein is a method for producing an amplified RNA product via use of a replicase expressed by the present recombinant vector.

### 2.1 Recombinant Vector of the Present Disclosure

The present disclosure aims at providing novel, synthetic deoxyribonucleic acids (DNAs) capable of encoding specific proteins. Accordingly, one aspect of the present disclosure is directed to a recombinant vector that comprises a polydeoxyribonucleotide of SEQ ID NOs: 1, 2 or 3. The recombinant vector has been optimized using human codons to enhance the expression of a replicase encoded therein.

The present novel deoxyribonucleic acid (DNA) sequences may be transcribed and translated into desired, functional proteins, specifically replicases that can be used during RNA amplification. Therefore, based on the primary structures of a designated protein, the present DNAs are designed and synthesized with the aid of tools and methods well-known in the art. Examples of tools and procedures widely used in the art for synthesizing the present novel DNA sequences include, but are not limited to, oligonucleotide synthesis (e.g., phosphoramidites method, and micro-array-based platforms), gene synthesis (e.g., oligonucleotide ligation assay, polymerase cycle assembly (PCA) assay, and etc.), CRISPR-Cas9 technology, transcription activator-like (TAL) effector nucleases (TALENs), next-generation sequencing (NGS), synthetic biology platforms (e.g., automated lab robots, genomic application programming interfaces (APIs), and etc.), directed evolution (DE), bioinformatics tools, (e.g., sequence alignment software including BLAST, ClustalW, and MUSCLE; genome browsers including Ensembl, UCSC Genome Browser, and IGV; structural biology software including PyMOL, Chimera, and VMD; protein structure prediction software including AlphaFold, HHpred, and I-TASSER), gene synthesis automation (e.g., automated DNA/RNA synthesizer, high-throughput cloning assembly, and artificial intelligence (AI)-powered systems), optimization algorithms (e.g., codon optimization, particle swarm optimization (PSO), cuckoos optimization algorithm (COA), whale optimization algorithm (WOA), ant colony optimization (ACO), simulated annealing (SA), Lion-AYAD, and etc.), and a combination thereof. According to some embodiments of the present disclosure, with the assistance of codon optimization procedures, three novel polydeoxynucleotide sequences of the present disclosure (i.e., SEQ ID NOs: 1, 2 and 3) are reversely synthesized based on the known amino acid sequences of nonstructural protein (nsp) 12, nsp 9, and nsp 14 of SARS-CoV2 virus, respectively. Examples of codon optimization procedures suitable for use in the present application include, but are not limited to, codon adaptation index (CAI), effective number of codons (ENC), GC content optimization, codon usage pair (CUP) optimization, context-dependent codon optimization (CDC), machine learning-based codon optimization, and a combination thereof. In one working example of the present disclosure, the codon optimization procedures are codon adaptation index (CAI) and/or machine learning-based codon optimization. According to alternative embodiments, the DNA sequences of the present disclosure can be transcribed into a ribonucleic acid (RNA) having a polyribonucleotide sequence of SEQ ID NOs: 4, 5, or 6.

According to some embodiments of the present disclosure, a recombinant vector is used to bear and express the present polydeoxyribonucleotide(s). The recombinant vector may be derived from plasmid or viral genomes, and typically comprises a promoter and regulatory elements, origin of replication (Ori), terminator sequence, and/or multiple cloning sites (MCS) independently and operably linked to the present polydeoxyribonucleotide(s), thus facilitates the expression of the present polydeoxyribonucleotide(s). Depending on the host (e.g., a competent cell or a cell-free expression system), selectable markers and reporter genes may be optionally or additionally constructed to the recombinant vector.

According to embodiments of the present disclosure, a cell-free protein expression system is used to express the present polydeoxyribonucleotide of SEQ ID NOs: 1, 2 or 3 carried by the recombinant vector in vitro. In working embodiments of the present disclosure, the expression of the present recombinant vector is achieved using commercially available cell-free protein expression kits or services.

According to some embodiments of the present disclosure, the replicase expressed through the present recombinant vector includes an amino acid sequence of SEQ ID NOs: 7, 8 or 9.

### 2.2 RNA Amplification Via Use of the Replicase Expressed by the Present Recombinant Vector

Another aspect of the present disclosure is directed to a method for producing an amplified RNA product from a nucleotide template in vitro with the aid of the replicases expressed by the present recombinant vector set forth in section 2.1 of this paper. According to embodiments of the present disclosure, the amplified RNA product is produced by an RNA cycling reaction (RCR), in which the present replicase encoded by the present polydeoxyribonucleotide (i.e., SEQ ID NOs: 1, 2 or 3) serves as an RNA-dependent RNA polymerase (RdRp) to replicate RNAs from an RNA template or a chimeric DNA/RNA oligonucleotide.

In some working embodiments of the present disclosure, the amplified RNA product is amplified from the RNA template, in most cases a sense-strand RNA sequence, encompasses at least one RdRp-binding sites recognizable by the present replicase(s). The amplification starts from binding of the present replicase(s) to the RdRp-binding sites of the RNA template, and the replication is carried out to produce a complementary, antisense-strand RNA sequence. The antisense-strand RNA sequence also encompasses the RNA template and the RdRp-binding site recognizable by the present replicase(s), thus will allow the replication to continue. Accordingly, multiple amplification cycles can be performed at predetermined conditions (e.g., temperatures and times) according to practical needs, and eventually the RNA template with desired sequences are replicated several times, thereby producing a desired, amplified RNA product.

According to embodiments of the present disclosure, the RNA template may be or contain any of desired polyribonucleotide sequences of a coding RNA or a non-coding RNA, as long as it is flanked by two RdRp binding sites recognizable for the present replicases and useful for producing RNA copies.

According to some embodiments of the present disclosure, the coding RNA includes a messenger RNA (mRNA) that encodes an antigen. Specifically, the coding RNA may be the mRNA that encodes a cancer antigen, a tumor antigen, a fungal antigen, a parasitic antigen, a bacterial antigen, a viral antigen, or a combination thereof.

Examples of the afore-mentioned tumor antigen include, but are not limited to, a neoantigen, a tumor-derived lysate, an alpha-fetoprotein (AFP), a carcinoembryonic antigen (CEA), a mucin protein, an epithelial tumor antigen (ETA), a tyrosinase, a melanoma-associated antigen (MAGE), a RAS protein, a tumor suppressor protein, and a combination thereof.

Examples of the afore-mentioned bacterial antigen include those derived from a bacterial species, which includes genera of Actinomyces, Aeromonas, Arthrobacter, Bacillus, Bacteroides, Bordetella, Borrelia, Brucella, Campylobacter, Chlamydia, Citrobacter, Clostridium, Corynebacterium, Escherichia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia, but not limited thereto.

Examples of the afore-mentioned viral antigen include those derived from a viral species, which includes but is not limited to, Adenovirus, Alphacoronavirus, Betacoronavirus, Cytomegalovirus, Deltainfluenzavirus, Deltacoronavirus, Gammacoronavirus, Hepacivirus, Hepatovirus, Influenza A virus, Influenza B virus, Influenza C virus, Influenza D virus, Lentivirus, Letovirus, Lymphocryptovirus, Orthopneumovirus, Orthopoxvirus, Papillomavirus, Quaranjavirus, Rotavirus, Simplexvirus, and Varicellovirus. In some working embodiments of the present disclosure, the viral antigen is expected to be derived from a Betacoronavirus genus; preferably is a spike(S) protein of Severe acute respiratory syndrome-related coronavirus (SARS-CoV-2).

Examples of the fungal antigen include those derived from a fungal species that causes a fungal infection, which includes but is not limited to, aspergillosis, blastomycosis, candidiasis, chromoblastomycosis, cryptococcosis, histoplasmosis, mycetoma, paracoccidioidomycosis, ringworm, tinea versicolor, and a combination thereof.

Examples of the afore-mentioned parasitic antigen include those derived from a parasite species that causes a parasitic infection including but not limited to, African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, trichuriasis, and a combination thereof.

According to embodiments of the present disclosure, the mRNA can be designed as a self-amplifying mRNA (saRNA) according to practical needs. Specifically, upstream of the desired RNA (e.g., the viral antigen RNA), the mRNA template can further include a polyribonucleotide sequence encoding RdRp polymerases. For instance, nonstructural proteins (nsP1-4) derived from viruses that are expected to form a complete RdRp protein can be included. In this way, the RdRp polymerases can be translated initially and, in turn, assist in the amplification of the mRNA. As a result, thus-designed mRNA possesses a self-amplification property and can be serve as the saRNA for versatile application.

According to embodiments of the present disclosure, the non-coding RNA that can serve as the RNA template includes, but is not limited to, a small interfering RNA (siRNA), a ribosomal RNA (rRNA), a transfer RNA (tRNA), a microRNA (miRNA), or an aptamer. Examples of the miRNA include, but are not limited to, a precursor miRNA or a mature miRNA.

Alternatively or optionally, the RNA template may be a chimeric DNA/RNA oligonucleotide. The chimeric DNA/RNA oligonucleotides can be artificially synthesized by tools and materials well known in the art. Nonetheless, in the RCR reaction set forth above, the progress of the reaction remains unhindered as long as the nucleotides serving as the template include at least one RdRp-binding sites recognizable by the present replicase. Whether the presence of polydeoxynucleotides in the template does not impede the advancement of RCR, allowing for successful RNA amplification.

### 2.3 Kit

The replicase of the present disclosure can be packaged into a kit along with other components required for RCR reaction. Thus, another aspect of the present disclosure is directed to a kit comprising at least one of the present replicases, which are expressed by the present recombinant vector as set forth above. In addition to the present replicases, components required for RCR reaction to be packaged in the kit include, but are not limited to, an RNA template, mixture of ribonucleoside triphosphate molecules (rNTPs), and reacting buffers (e.g., transcription buffer). The individual components of the present kit can be packaged in separate containers. Example of packaging materials or containers suitable for use in the present kit includes, but is not limited to, vials, bottles, jars, flexible packaging (e.g., sealed Mylar or plastic bags), and the like. Other reagents can be included in separate containers and provided with the kit; e.g., positive control samples, negative control samples, and/or buffers.

According to some embodiments of the present disclosure, the kit can comprise instructions for use in accordance with any of the methods described herein. The instructions can comprise a description of protocol and conditions for the operating of RCR reaction to produce an amplified RNA product.

According to some embodiments of the present disclosure, the label or package insert may indicate that the component and the present replicase is used for RCR procedure described herein.

### EXAMPLES

### Materials and Methods

### Codon Optimization

Codon optimization algorithms were utilized to design three polydeoxyribonucleotides based on three nonstructural proteins of SARS-CoV-2, nsp12, nsp9, and nsp14, respectively, using publicly available codon optimization tools.

### Construction of Recombinant Vector in Cell-Free Expression System

All standard reagents of a cell-free expression system, an in vitro protein synthesis kit, and all molecular biology reagents were obtained from commercial suppliers. To construct the present recombinant vector, the present polydeoxyribonucleotides were modified with a C-terminal 10× Histidine tag (10×His) in a pET28a vector in accordance with standard procedures provided by the supplier. To express the replicases, the thus-produced recombinant vector was further modified to include a C-terminal V5 tag (14aa) with a GSSG linker between the coding regions and the 10×His tag for use in cell-free expression systems.

### RNA-Dependent RNA Cycling Reaction (RCR)

The RNA template (0.01 ng-10 μg), the present replicases (0.1-50 U), and rNTPs were mixed in 1× transcription buffer containing Tris-HCl buffer supplemented with MgCl₂, NaCl, spermidine, TMG, DMSO, and/or MOPS (0.001-10 mM), thereby forming a reaction mixture. The reaction mixture was subjected to RNA-dependent RCR by incubating at 20-45° C. for 1-6 hours, thereby producing amplified RNA products. The quantity of the amplified RNA products was then confirmed using agarose gel electrophoresis.

### Example 1: Synthesis of the Present Polydeoxyribonucleotides

The present polydeoxyribonucleotides were designed and synthesized based on three nonstructural proteins by methods described in the “Materials and Methods” section. The thus-produced polydeoxyribonucleotides RdRP-1, RdRP-2, and RdRP-3, and their sequences are listed in Tables 1 and 2, respectively.

TABLE 1
The present polydeoxyribonucleotides

| Polydeoxyrib | | | SEQ |
|---|---|---|---|
| onucleotides | DNA sequence (5′ to 3′) | Length | ID NO |
| RdRP-1 | ATGCAATCATTTTTGAACCGTGTTTGCGGAGTG | 2790 bps | 1 |
| (Nsp 12) | TCCGCTGCACGTCTCACGCCTTGTGGTACGGGG | | |
| | ACGTCTACCGATGTGGTGTACCGGGCGTTCGAC | | |
| | ATCTACAACGATAAGGTGGCTGGATTCGCCAA | | |
| | GTTTCTCAAAACCAACTGTTGTAGATTTCAGGA | | |
| | GAAGGACGAGGATGATAACTTGATAGACAGTT | | |
| | ACTTTGTGGTGAAAAGACATACATTTAGCAATT | | |
| | ACCAGCACGAAGAAACGATATATAATCTGCTG | | |
| | AAAGATTGCCCTGCCGTTGCTAAGCACGACTTT | | |
| | TTTAAGTTTCGCATCGACGGCGACATGGTGCCC | | |
| | CATATTAGTCGACAACGTCTCACTAAGTACACC | | |
| | ATGGCAGACCTGGTGTACGCCCTGAGGCACTTT | | |
| | GACGAGGGGAACTGTGATACATTGAAGGAAAT | | |
| | TCTCGTCACCTACAACTGCTGTGATGACGACTA | | |
| | TTTCAACAAAAAAGATTGGTATGACTTTGTTGA | | |
| | GAACCCAGATATTCTCCGCGTGTACGCCAATCT | | |
| | GGGGGAGAGAGTCCGTCAAGCCTTACTTAAAA | | |
| | CAGTCCAGTTCTGTGACGCAATGAGAAACGCG | | |
| | GGAATCGTTGGCGTGCTGACATTAGATAACCA | | |
| | GGATCTTAATGGCAATTGGTATGACTTCGGGGA | | |
| | CTTTATCCAAACAACCCCTGGCAGCGGCGTTCC | | |
| | CGTCGTCGATTCCTACTATAGTCTCTTAATGCC | | |
| | AATTCTGACTCTGACTAGAGCCTTAACCGCCGA | | |
| | GAGTCATGTCGACACAGATTTGACAAAGCCCT | | |
| | ACATAAAGTGGGATCTCCTTAAATATGATTTTA | | |
| | CTGAAGAACGACTGAAGCTTTTCGACCGCTATT | | |
| | TCAAATATTGGGACCAAACTTATCACCCTAATT | | |
| | GTGTGAATTGCTTAGATGATCGCTGCATTCTTC | | |
| | ATTGCGCTAATTTCAACGTGTTATTCTCAACCG | | |
| | TGTTTCCTCCTACTTCCTTCGGCCCATTGGTGAG | | |
| | GAAAATTTTTGTCGATGGTGTCCCCTTTGTGGT | | |
| | GTCAACAGGCTATCACTTTAGGGAATTGGGGGT | | |
| | CGTCCATAACCAGGATGTCAATTTGCATTCCTC | | |
| | TCGGCTCTCCTTCAAGGAGCTGCTGGTGTATGC | | |
| | CGCGGACCCCGCTATGCACGCCGCCAGCGGAA | | |
| | ACCTCCTCCTGGACAAGAGGACAACGTGTTTTT | | |
| | CCGTAGCAGCTCTTACCAACAACGTTGCCTTCC | | |
| | AGACAGTTAAACCCGGAAACTTTAATAAAGAC | | |
| | TTCTATGATTTTGCTGTTTCCAAAGGCTTCTTCA | | |
| | AGGAGGGGTCATCAGTGGAGCTGAAGCATTTC | | |
| | TTCTTCGCTCAGGACGGCAATGCGGCGATTTCT | | |
| | GATTATGATTATTACCGCTACAATCTGCCCACA | | |
| | ATGTGTGATATCAGACAGTTATTATTTGTGGTG | | |
| | GAAGTTGTCGACAAGTATTTCGACTGCTACGAT | | |
| | GGAGGTTGCATCAACGCAAACCAAGTGATTGT | | |
| | TAATAACCTGGACAAGAGTGCAGGCTTCCCTTT | | |
| | TAACAAGTGGGGCAAGGCCAGACTCTACTATG | | |
| | ACTCCATGTCTTATGAGGATCAGGACGCTCTGT | | |
| | TTGCGTACACAAAGCGCAATGTTATCCCAACGA | | |
| | TTACCCAAATGAATCTGAAATACGCTATCTCTG | | |
| | CTAAGAATAGAGCTCGTACCGTCGCTGGAGTTT | | |
| | CAATCTGCAGCACAATGACTAATAGGCAGTTCC | | |
| | ATCAAAAACTGCTTAAGAGCATAGCGGCTACA | | |
| | CGTGGAGCAACTGTCGTGATTGGGACTAGTAA | | |
| | GTTCTATGGCGGGTGGCACAACATGCTCAAGA | | |
| | CGGTATACTCCGATGTAGAAAATCCCCACCTCA | | |
| | TGGGATGGGATTACCCCAAATGCGATAGAGCA | | |
| | ATGCCGAACATGCTCCGGATTATGGCTTCTCTT | | |
| | GTGCTTGCACGAAAACATACAACCTGTTGTAGC | | |
| | CTGTCCCACCGGTTCTATAGACTCGCTAATGAG | | |
| | TGCGCCCAGGTTCTCAGTGAAATGGTTATGTGC | | |
| | GGGGGCTCTCTGTATGTAAAACCTGGCGGCACC | | |
| | AGCAGTGGCGATGCCACAACTGCCTATGCAAA | | |
| | CTCCGTGTTTAACATTTGCCAAGCCGTGACGGC | | |
| | AAACGTGAACGCTCTTTTGTCAACCGACGGCAA | | |
| | TAAAATCGCCGACAAGTATGTCAGAAACCTCC | | |
| | AGCATCGGCTGTATGAGTGTCTGTACCGCAACC | | |
| | GGGATGTGGACACTGATTTTGTGAACGAATTCT | | |
| | ATGCATACTTGAGGAAACACTTCAGCATGATG | | |
| | ATCCTCTCCGATGACGCTGTGGTGTGCTTTAAC | | |
| | TCAACATATGCATCTCAAGGACTCGTTGCGTCC | | |
| | ATTAAAAATTTTAAGTCTGTTCTCTATTACCAG | | |
| | AACAACGTATTCATGTCCGAAGCTAAATGCTGG | | |
| | ACCGAGACTGACTTGACCAAGGGCCCACACGA | | |
| | ATTTTGCTCCCAACATACCATGCTCGTCAAGCA | | |
| | AGGGGACGATTATGTGTATCTCCCATACCCGGA | | |
| | TCCATCACGGATTTTGGGAGCTGGGTGCTTTGT | | |
| | GGATGATATCGTGAAGACCGATGGCACCCTGA | | |
| | TGATCGAAAGATTCGTGTCACTTGCCATCGATG | | |
| | CTTATCCTTTGACGAAACACCCAAACCAGGAAT | | |
| | ACGCCGACGTCTTCCATCTGTATCTGCAGTACA | | |
| | TTCGCAAACTGCACGACGAACTCACAGGCCAC | | |
| | ATGCTGGACATGTACTCAGTGATGCTCACAAAC | | |
| | GATAACACATCCCGGTACTGGGAGCCCGAGTTT | | |
| | TACGAAGCGATGTACACCCCGCATACTGTGCTG | | |
| | CAGTAA | | |
| RdRP-2 | ATGAATAACGAGCTGAGCCCTGTTGCACTGAG | 345 bps | 2 |
| (Nsp 9) | GCAGATGAGCTGCGCTGCCGGTACGACACAGA | | |
| | CAGCTTGCACCGATGATAATGCCTTGGCCTATT | | |
| | ACAATACTACTAAAGGTGGCCGTTTCGTCCTGG | | |
| | CGTTACTGAGCGATCTGCAGGATCTCAAGTGGG | | |
| | CTCGTTTTCCCAAGTCAGACGGTACTGGCACCA | | |
| | TTTATACAGAACTGGAGCCACCGTGCAGATTTG | | |
| | TAACAGATACCCCCAAGGGGCCTAAGGTTAAA | | |
| | TACCTGTACTTCATTAAGGGACTCAATAACCTG | | |
| | AACAGAGGCATGGTGCTCGGTTCCCTCGCCGCT | | |
| | ACAGTGCGGCTCCAGTGA | | |
| RdRP-3 | ATGGCCGAGAATGTGACCGGATTGTTCAAGGA | 1587 bps | 3 |
| (Nsp 14) | TTGTAGTAAGGTCATCACCGGTCTCCATCCTAC | | |
| | CCAGGCACCGACCCACTTGTCTGTCGATACCAA | | |
| | GTTTAAGACCGAGGGCCTGTGCGTGGACATAC | | |
| | CAGGAATTCCTAAGGATATGACTTATCGCCGTT | | |
| | TGATTTCCATGATGGGCTTCAAGATGAACTACC | | |
| | AGGTCAACGGTTATCCTAATATGTTCATCACCC | | |
| | GCGAGGAGGCCATTAGACACGTAAGAGCTTGG | | |
| | ATAGGATTCGATGTAGAAGGCTGTCATGCTACC | | |
| | AGAGAAGCCGTGGGTACCAACCTGCCGCTGCA | | |
| | GCTTGGCTTCAGCACTGGCGTGAATCTGGTGGC | | |
| | GGTGCCGACCGGTTATGTGGATACCCCTAACAA | | |
| | CACAGACTTCTCCCGCGTATCTGCTAAACCCCC | | |
| | CCCTGGCGACCAGTTTAAGCACCTGATCCCTCT | | |
| | CATGTATAAGGGACTTCCCTGGAATGTGGTGAG | | |
| | GATCAAAATTGTTCAAATGTTGAGCGACACCCT | | |
| | TAAGAACCTTAGTGATAGGGTCGTCTTCGTCCT | | |
| | CTGGGCTCACGGATTTGAACTCACAAGCATGA | | |
| | AGTATTTTGTCAAAATTGGGCCAGAGCGAACAT | | |
| | GTTGCCTTTGCGACAGACGTGCTACTTGCTTTT | | |
| | CTACCGCTTCCGACACCTATGCCTGCTGGCACC | | |
| | ACTCTATCGGTTTTGATTACGTATACAATCCTTT | | |
| | TATGATCGACGTGCAGCAGTGGGGCTTTACAG | | |
| | GCAATCTTCAGTCAAACCACGACCTTTATTGCC | | |
| | AGGTACACGGCAATGCCCATGTCGCCTCCTGCG | | |
| | ATGCCATCATGACTCGTTGTCTCGCAGTCCACG | | |
| | AATGTTTTGTGAAGAGAGTTGATTGGACGATCG | | |
| | AATATCCAATCATTGGTGATGAGTTAAAAATAA | | |
| | ACGCTGCTTGCCGCAAGGTGCAGCATATGGTG | | |
| | GTGAAGGCAGCCCTCTTAGCAGATAAGTTTCCA | | |
| | GTGCTGCACGACATAGGAAACCCTAAGGCCAT | | |
| | TAAGTGTGTCCCCCAAGCGGACGTGGAGTGGA | | |
| | AATTCTACGACGCACAGCCCTGCAGTGACAAA | | |
| | GCCTACAAGATTGAGGAGCTCTTCTATTCATAC | | |
| | GCCACACATAGTGACAAGTTTACAGATGGAGT | | |
| | CTGTCTGTTTTGGAACTGCAATGTGGACAGATA | | |
| | CCCCGCTAATAGCATCGTTTGTCGTTTCGACAC | | |
| | AAGGGTTCTGAGCAACCTTAATCTTCCAGGCTG | | |
| | CGACGGGGGATCCTTATACGTTAACAAACACG | | |
| | CGTTTCACACCCCTGCCTTTGATAAATCCGCTTT | | |
| | TGTGAACCTGAAACAATTACCGTTTTTCTACTA | | |
| | TAGTGACTCTCCTTGCGAGTCCCACGGGAAGCA | | |
| | GGTCGTGTCAGACATAGACTACGTGCCACTGA | | |
| | AGTCAGCTACGTGCATAACTCGGTGCAATTTAG | | |
| | GAGGCGCTGTGTGTCGGCATCATGCTAACGAAT | | |
| | ACCGGCTGTACCTGGACGCATACAACATGATG | | |
| | ATCAGCGCCGGATTTTCTTTGTGGGTTTACAAG | | |
| | CAGTTTGACACATATAACCTGTGGAATACATTC | | |
| | ACGAGACTCCAGTGA | | |

TABLE 2
The present polyribonucleotide sequences transcribed from the
present polydeoxyribonucleotides in Table 1

| Polyribo- | | | SEQ |
|---|---|---|---|
| nucleotides | RNA sequence (5′ to 3′) | Length | ID NO |
| RdRP-1 | AUGCAAUCAUUUUUGAACCGUGUUUGCGGAG | 2790 bps | 4 |
| (Nsp 12) | UGUCCGCUGCACGUCUCACGCCUUGUGGUACG | | |
| | GGGACGUCUACCGAUGUGGUGUACCGGGCGU | | |
| | UCGACAUCUACAACGAUAAGGUGGCUGGAUU | | |
| | CGCCAAGUUUCUCAAAACCAACUGUUGUAGA | | |
| | UUUCAGGAGAAGGACGAGGAUGAUAACUUGA | | |
| | UAGACAGUUACUUUGUGGUGAAAAGACAUAC | | |
| | AUUUAGCAAUUACCAGCACGAAGAAACGAUA | | |
| | UAUAAUCUGCUGAAAGAUUGCCCUGCCGUUG | | |
| | CUAAGCACGACUUUUUUAAGUUUCGCAUCGA | | |
| | CGGCGACAUGGUGCCCCAUAUUAGUCGACAAC | | |
| | GUCUCACUAAGUACACCAUGGCAGACCUGGU | | |
| | GUACGCCCUGAGGCACUUUGACGAGGGGAAC | | |
| | UGUGAUACAUUGAAGGAAAUUCUCGUCACCU | | |
| | ACAACUGCUGUGAUGACGACUAUUUCAACAA | | |
| | AAAAGAUUGGUAUGACUUUGUUGAGAACCCA | | |
| | GAUAUUCUCCGCGUGUACGCCAAUCUGGGGG | | |
| | AGAGAGUCCGUCAAGCCUUACUUAAAACAGU | | |
| | CCAGUUCUGUGACGCAAUGAGAAACGCGGGA | | |
| | AUCGUUGGCGUGCUGACAUUAGAUAACCAGG | | |
| | AUCUUAAUGGCAAUUGGUAUGACUUCGGGGA | | |
| | CUUUAUCCAAACAACCCCUGGCAGCGGCGUUC | | |
| | CCGUCGUCGAUUCCUACUAUAGUCUCUUAAU | | |
| | GCCAAUUCUGACUCUGACUAGAGCCUUAACCG | | |
| | CCGAGAGUCAUGUCGACACAGAUUUGACAAA | | |
| | GCCCUACAUAAAGUGGGAUCUCCUUAAAUAU | | |
| | GAUUUUACUGAAGAACGACUGAAGCUUUUCG | | |
| | ACCGCUAUUUCAAAUAUUGGGACCAAACUUA | | |
| | UCACCCUAAUUGUGUGAAUUGCUUAGAUGAU | | |
| | CGCUGCAUUCUUCAUUGCGCUAAUUUCAACG | | |
| | UGUUAUUCUCAACCGUGUUUCCUCCUACUUCC | | |
| | UUCGGCCCAUUGGUGAGGAAAAUUUUUGUCG | | |
| | AUGGUGUCCCCUUUGUGGUGUCAACAGGCUA | | |
| | UCACUUUAGGGAAUUGGGGGUCGUCCAUAAC | | |
| | CAGGAUGUCAAUUUGCAUUCCUCUCGGCUCUC | | |
| | CUUCAAGGAGCUGCUGGUGUAUGCCGCGGAC | | |
| | CCCGCUAUGCACGCCGCCAGCGGAAACCUCCU | | |
| | CCUGGACAAGAGGACAACGUGUUUUUCCGUA | | |
| | GCAGCUCUUACCAACAACGUUGCCUUCCAGAC | | |
| | AGUUAAACCCGGAAACUUUAAUAAAGACUUC | | |
| | UAUGAUUUUGCUGUUUCCAAAGGCUUCUUCA | | |
| | AGGAGGGGUCAUCAGUGGAGCUGAAGCAUUU | | |
| | CUUCUUCGCUCAGGACGGCAAUGCGGCGAUU | | |
| | UCUGAUUAUGAUUAUUACCGCUACAAUCUGC | | |
| | CCACAAUGUGUGAUAUCAGACAGUUAUUAUU | | |
| | UGUGGUGGAAGUUGUCGACAAGUAUUUCGAC | | |
| | UGCUACGAUGGAGGUUGCAUCAACGCAAACC | | |
| | AAGUGAUUGUUAAUAACCUGGACAAGAGUGC | | |
| | AGGCUUCCCUUUUAACAAGUGGGGCAAGGCC | | |
| | AGACUCUACUAUGACUCCAUGUCUUAUGAGG | | |
| | AUCAGGACGCUCUGUUUGCGUACACAAAGCG | | |
| | CAAUGUUAUCCCAACGAUUACCCAAAUGAAU | | |
| | CUGAAAUACGCUAUCUCUGCUAAGAAUAGAG | | |
| | CUCGUACCGUCGCUGGAGUUUCAAUCUGCAGC | | |
| | ACAAUGACUAAUAGGCAGUUCCAUCAAAAAC | | |
| | UGCUUAAGAGCAUAGCGGCUACACGUGGAGC | | |
| | AACUGUCGUGAUUGGGACUAGUAAGUUCUAU | | |
| | GGCGGGUGGCACAACAUGCUCAAGACGGUAU | | |
| | ACUCCGAUGUAGAAAAUCCCCACCUCAUGGGA | | |
| | UGGGAUUACCCCAAAUGCGAUAGAGCAAUGC | | |
| | CGAACAUGCUCCGGAUUAUGGCUUCUCUUGU | | |
| | GCUUGCACGAAAACAUACAACCUGUUGUAGC | | |
| | CUGUCCCACCGGUUCUAUAGACUCGCUAAUGA | | |
| | GUGCGCCCAGGUUCUCAGUGAAAUGGUUAUG | | |
| | UGCGGGGGCUCUCUGUAUGUAAAACCUGGCG | | |
| | GCACCAGCAGUGGCGAUGCCACAACUGCCUAU | | |
| | GCAAACUCCGUGUUUAACAUUUGCCAAGCCG | | |
| | UGACGGCAAACGUGAACGCUCUUUUGUCAAC | | |
| | CGACGGCAAUAAAAUCGCCGACAAGUAUGUC | | |
| | AGAAACCUCCAGCAUCGGCUGUAUGAGUGUC | | |
| | UGUACCGCAACCGGGAUGUGGACACUGAUUU | | |
| | UGUGAACGAAUUCUAUGCAUACUUGAGGAAA | | |
| | CACUUCAGCAUGAUGAUCCUCUCCGAUGACGC | | |
| | UGUGGUGUGCUUUAACUCAACAUAUGCAUCU | | |
| | CAAGGACUCGUUGCGUCCAUUAAAAAUUUUA | | |
| | AGUCUGUUCUCUAUUACCAGAACAACGUAUU | | |
| | CAUGUCCGAAGCUAAAUGCUGGACCGAGACU | | |
| | GACUUGACCAAGGGCCCACACGAAUUUUGCUC | | |
| | CCAACAUACCAUGCUCGUCAAGCAAGGGGACG | | |
| | AUUAUGUGUAUCUCCCAUACCCGGAUCCAUCA | | |
| | CGGAUUUUGGGAGCUGGGUGCUUUGUGGAUG | | |
| | AUAUCGUGAAGACCGAUGGCACCCUGAUGAU | | |
| | CGAAAGAUUCGUGUCACUUGCCAUCGAUGCU | | |
| | UAUCCUUUGACGAAACACCCAAACCAGGAAU | | |
| | ACGCCGACGUCUUCCAUCUGUAUCUGCAGUAC | | |
| | AUUCGCAAACUGCACGACGAACUCACAGGCCA | | |
| | CAUGCUGGACAUGUACUCAGUGAUGCUCACA | | |
| | AACGAUAACACAUCCCGGUACUGGGAGCCCGA | | |
| | GUUUUACGAAGCGAUGUACACCCCGCAUACU | | |
| | GUGCUGCAGUAA | | |
| RdRP-2 | AUGAAUAACGAGCUGAGCCCUGUUGCACUGA | 345 bps | 5 |
| (Nsp 9) | GGCAGAUGAGCUGCGCUGCCGGUACGACACA | | |
| | GACAGCUUGCACCGAUGAUAAUGCCUUGGCC | | |
| | UAUUACAAUACUACUAAAGGUGGCCGUUUCG | | |
| | UCCUGGCGUUACUGAGCGAUCUGCAGGAUCU | | |
| | CAAGUGGGCUCGUUUUCCCAAGUCAGACGGU | | |
| | ACUGGCACCAUUUAUACAGAACUGGAGCCACC | | |
| | GUGCAGAUUUGUAACAGAUACCCCCAAGGGG | | |
| | CCUAAGGUUAAAUACCUGUACUUCAUUAAGG | | |
| | GACUCAAUAACCUGAACAGAGGCAUGGUGCU | | |
| | CGGUUCCCUCGCCGCUACAGUGCGGCUCCAGU | | |
| | GA | | |
| RdRP-3 | AUGGCCGAGAAUGUGACCGGAUUGUUCAAGG | 1587 bps | 6 |
| (Nsp 14) | AUUGUAGUAAGGUCAUCACCGGUCUCCAUCC | | |
| | UACCCAGGCACCGACCCACUUGUCUGUCGAUA | | |
| | CCAAGUUUAAGACCGAGGGCCUGUGCGUGGA | | |
| | CAUACCAGGAAUUCCUAAGGAUAUGACUUAU | | |
| | CGCCGUUUGAUUUCCAUGAUGGGCUUCAAGA | | |
| | UGAACUACCAGGUCAACGGUUAUCCUAAUAU | | |
| | GUUCAUCACCCGCGAGGAGGCCAUUAGACACG | | |
| | UAAGAGCUUGGAUAGGAUUCGAUGUAGAAGG | | |
| | CUGUCAUGCUACCAGAGAAGCCGUGGGUACC | | |
| | AACCUGCCGCUGCAGCUUGGCUUCAGCACUGG | | |
| | CGUGAAUCUGGUGGCGGUGCCGACCGGUUAU | | |
| | GUGGAUACCCCUAACAACACAGACUUCUCCCG | | |
| | CGUAUCUGCUAAACCCCCCCCUGGCGACCAGU | | |
| | UUAAGCACCUGAUCCCUCUCAUGUAUAAGGG | | |
| | ACUUCCCUGGAAUGUGGUGAGGAUCAAAAUU | | |
| | GUUCAAAUGUUGAGCGACACCCUUAAGAACC | | |
| | UUAGUGAUAGGGUCGUCUUCGUCCUCUGGGC | | |
| | UCACGGAUUUGAACUCACAAGCAUGAAGUAU | | |
| | UUUGUCAAAAUUGGGCCAGAGCGAACAUGUU | | |
| | GCCUUUGCGACAGACGUGCUACUUGCUUUUC | | |
| | UACCGCUUCCGACACCUAUGCCUGCUGGCACC | | |
| | ACUCUAUCGGUUUUGAUUACGUAUACAAUCC | | |
| | UUUUAUGAUCGACGUGCAGCAGUGGGGCUUU | | |
| | ACAGGCAAUCUUCAGUCAAACCACGACCUUUA | | |
| | UUGCCAGGUACACGGCAAUGCCCAUGUCGCCU | | |
| | CCUGCGAUGCCAUCAUGACUCGUUGUCUCGCA | | |
| | GUCCACGAAUGUUUUGUGAAGAGAGUUGAUU | | |
| | GGACGAUCGAAUAUCCAAUCAUUGGUGAUGA | | |
| | GUUAAAAAUAAACGCUGCUUGCCGCAAGGUG | | |
| | CAGCAUAUGGUGGUGAAGGCAGCCCUCUUAG | | |
| | CAGAUAAGUUUCCAGUGCUGCACGACAUAGG | | |
| | AAACCCUAAGGCCAUUAAGUGUGUCCCCCAAG | | |
| | CGGACGUGGAGUGGAAAUUCUACGACGCACA | | |
| | GCCCUGCAGUGACAAAGCCUACAAGAUUGAG | | |
| | GAGCUCUUCUAUUCAUACGCCACACAUAGUG | | |
| | ACAAGUUUACAGAUGGAGUCUGUCUGUUUUG | | |
| | GAACUGCAAUGUGGACAGAUACCCCGCUAAU | | |
| | AGCAUCGUUUGUCGUUUCGACACAAGGGUUC | | |
| | UGAGCAACCUUAAUCUUCCAGGCUGCGACGG | | |
| | GGGAUCCUUAUACGUUAACAAACACGCGUUU | | |
| | CACACCCCUGCCUUUGAUAAAUCCGCUUUUGU | | |
| | GAACCUGAAACAAUUACCGUUUUUCUACUAU | | |
| | AGUGACUCUCCUUGCGAGUCCCACGGGAAGCA | | |
| | GGUCGUGUCAGACAUAGACUACGUGCCACUG | | |
| | AAGUCAGCUACGUGCAUAACUCGGUGCAAUU | | |
| | UAGGAGGCGCUGUGUGUCGGCAUCAUGCUAA | | |
| | CGAAUACCGGCUGUACCUGGACGCAUACAACA | | |
| | UGAUGAUCAGCGCCGGAUUUUCUUUGUGGGU | | |
| | UUACAAGCAGUUUGACACAUAUAACCUGUGG | | |
| | AAUACAUUCACGAGACUCCAGUGA | | |

### Example 2: Replicases Expressed from the Present Polydeoxyribonucleotides of Example 1

The present replicases nsp12, nsp9, and nsp14 were respectively expressed by three recombinant vectors individually comprising the present deoxyribonucleotides RdRP-1, RdRP-2, and RdRP-3 of Example 1 (Table 1) in a cell-free expression system in accordance with the procedures described in the “Materials and Methods” section. The amino acid sequences of the present replicases are listed in Table 3.

TABLE 3
Amino acid sequences of the present replicases

| | | | SEQ |
|---|---|---|---|
| Replicase | Amino acid sequence (N′ to C′) | Length | ID NO |
| Nsp 12 | MQSFLNRVCGVSAARLTPCGTGTSTDVVYRAFDIY | 929 aa | 7 |
| | NDKVAGFAKFLKTNCCRFQEKDEDDNLIDSYFVV | | |
| | KRHTFSNYQHEETIYNLLKDCPAVAKHDFFKFRID | | |
| | GDMVPHISRQRLTKYTMADLVYALRHFDEGNCDT | | |
| | LKEILVTYNCCDDDYFNKKDWYDFVENPDILRVY | | |
| | ANLGERVRQALLKTVQFCDAMRNAGIVGVLTLDN | | |
| | QDLNGNWYDFGDFIQTTPGSGVPVVDSYYSLLMPI | | |
| | LTLTRALTAESHVDTDLTKPYIKWDLLKYDFTEER | | |
| | LKLFDRYFKYWDQTYHPNCVNCLDDRCILHCANF | | |
| | NVLFSTVFPPTSFGPLVRKIFVDGVPFVVSTGYHFR | | |
| | ELGVVHNQDVNLHSSRLSFKELLVYAADPAMHAA | | |
| | SGNLLLDKRTTCFSVAALTNNVAFQTVKPGNFNK | | |
| | DFYDFAVSKGFFKEGSSVELKHFFFAQDGNAAISD | | |
| | YDYYRYNLPTMCDIRQLLFVVEVVDKYFDCYDGG | | |
| | CINANQVIVNNLDKSAGFPFNKWGKARLYYDSMS | | |
| | YEDQDALFAYTKRNVIPTITQMNLKYAISAKNRAR | | |
| | TVAGVSICSTMTNRQFHQKLLKSIAATRGATVVIG | | |
| | TSKFYGGWHNMLKTVYSDVENPHLMGWDYPKCD | | |
| | RAMPNMLRIMASLVLARKHTTCCSLSHRFYRLAN | | |
| | ECAQVLSEMVMCGGSLYVKPGGTSSGDATTAYAN | | |
| | SVFNICQAVTANVNALLSTDGNKIADKYVRNLQH | | |
| | RLYECLYRNRDVDTDFVNEFYAYLRKHFSMMILS | | |
| | DDAVVCFNSTYASQGLVASIKNFKSVLYYQNNVF | | |
| | MSEAKCWTETDLTKGPHEFCSQHTMLVKQGDDY | | |
| | VYLPYPDPSRILGAGCFVDDIVKTDGTLMIERFVSL | | |
| | AIDAYPLTKHPNQEYADVFHLYLQYIRKLHDELTG | | |
| | HMLDMYSVMLTNDNTSRYWEPEFYEAMYTPHTV | | |
| | LQ | | |
| Nsp 9 | MNNELSPVALRQMSCAAGTTQTACTDDNALAYY | 114 aa | 8 |
| | NTTKGGRFVLALLSDLQDLKWARFPKSDGTGTIYT | | |
| | ELEPPCRFVTDTPKGPKVKYLYFIKGLNNLNRGMV | | |
| | LGSLAATVRLQ | | |
| Nsp 14 | MEGLCVDIPGIPKDMTYRRLISMMGFKMNYQVNG | 551 aa | 9 |
| | YPNMFITREEAIRHVRAWIGFDVEGEGLCVDIPGIP | | |
| | KDMTYRRLISMMGFKMNYQVNGYPNMFITREEAI | | |
| | RHVRAWIGFDVEGCHATREAVGTNLPLQLGFSTG | | |
| | VNLVAVPTGYVDTPNNTDFSRVSAKPPPGDQFKHL | | |
| | IPLMYKGLPWNVVRIKIVQMLSDTLKNLSDRVVFV | | |
| | LWAHGFELTSMKYFVKIGPERTCCLCDRRATCFST | | |
| | ASDTYACWHHSIGFDYVYNPFMIDVQQWGFTGNL | | |
| | QSNHDLYCQVHGNAHVASCDAIMTRCLAVHECFV | | |
| | KRVDWTIEYPIIGDELKINAACRKVQHMVVKAALL | | |
| | ADKFPVLHDIGNPKAIKCVPQADVEWKFYDAQPC | | |
| | SDKAYKIEELFYSYATHSDKFTDGVCLFWNCNVD | | |
| | RYPANSIVCRFDTRVLSNLNLPGCDGGSLYVNKHA | | |
| | FHTPAFDKSAFVNLKQLPFFYYSDSPCESHGKQVV | | |
| | SDIDYVPLKSATCITRQNLGGAVCRHHANEYRLYL | | |
| | DAYNMMISAGFSLWVYKQFDTYNLWNTFTRLQ | | |

### Example 3: Producing Amplified RNAs in the Presence of Replicases of Example 2

3.1 Amplification of the RNA of Enhanced Green Fluorescent Protein (eGFP)

In this example, whether the present replicases can effectively amplify RNA through the RNA-dependent RCR process was validated. To this purpose, a synthetic fragment of RNA template encoding enhanced green fluorescent protein (eGFP) was amplified through the RNA-dependent RCR procedure described in “Materials and Methods” section, in which the amplification was performed in the presence of a replicase mixture of nsp12, nsp9 and nsp14 respectively encoded by the present RdRP-1, RdRP-2, and RdRP-3 in according to the ratios specified in Table 4. The concentration of the amplified product (eGFP mRNA) was quantified through gel electrophoresis to assess the amplification efficiency of each replicase.

TABLE 4
Ratio of the present replicases in the mixture

| Mixture | I | II | III | IV | V | VI | VII | VIII |
|---|---|---|---|---|---|---|---|---|
| nsp12 | 1 | 1 | 0 | 0 | 2 | 1 | 1 | 1 |
| nsp9 | 1 | 0 | 1 | 0 | 1 | 2 | 1 | 2 |
| nsp14 | 1 | 0 | 0 | 1 | 1 | 1 | 2 | 2 |

It was found that, among the three replicases, RdRP-1 alone could achieve the highest amplification efficiency. Further, the combinational use of all three replicases (i.e., Mixtures I, V-VIII) also gave rise to high amplification efficiency (data not shown).

3.2 Amplification of the mRNA of SARS-CoV-2 Spike Protein

In this example, whether an RNA template capable of translating the SARS-CoV-2 spike protein may be amplified in the presence of the replicases of Example 3.1 in RNA-dependent RCR is investigated. To this purpose, the RNA template capable of translating the SARS-CoV-2 spike protein is amplified in the presence of Mixtures I, V, or VIII of Example 3.1, and the yield of the amplified product (spike protein mRNA) is quantified by gel electrophoresis.

It is expected to find that, the amplification is successfully accomplished in the present replicases, and the amplified RNA product is produced with a high purity ratio.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant vector for expressing a replicase comprising a polydeoxyribonucleotide of SEQ ID NOs: 1, 2 or 3.

2. The recombinant vector of claim 1, wherein the Poly deoxyribonucleic acid of SEQ ID NOs: 1, 2 or 3 is independently transcribed to a polyribonucleotide sequence of SEQ ID NOs: 4, 5, or 6.

3. The recombinant vector of claim 1, wherein the replicase comprises an amino acid sequence of SEQ ID NOs: 7, 8 or 9.

4. A method for producing an amplified RNA product in an RNA cycling reaction (RCR) comprising amplifying an RNA template in the presence of a replicase expressed by the recombinant vector of claim 1.

5. The method of claim 4, wherein the RNA template comprising a polyribonucleotide sequence of a coding RNA or a non-coding RNA.

6. The method of claim 5, wherein the coding RNA is a messenger RNA (mRNA) that encodes an antigen.

7. The method of claim 6, wherein the antigen is a cancer antigen, a tumor antigen, a bacterial antigen, a viral antigen, a fungal antigen, a parasitic antigen, or a combination thereof.

8. The method of claim 7, wherein the tumor antigen is selected from the group consisting of a neoantigen, a tumor-derived lysate, an alpha-fetoprotein (AFP), a carcinoembryonic antigen (CEA), a mucin protein, an epithelial tumor antigen (ETA), a tyrosinase, a melanoma-associated antigen (MAGE), a RAS protein, and a tumor suppressor protein.

9. The method of claim 7, wherein the bacterial antigen is derived from a bacterial species selected from the group consisting of Actinomyces, Aeromonas, Arthrobacter, Bacillus, Bacteroides, Bordetella, Borrelia, Brucella, Campylobacter, Chlamydia, Citrobacter, Clostridium, Corynebacterium, Escherichia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia.

10. The method of claim 7, wherein the viral antigen is derived from a viral species selected from the group consisting of Adenovirus, Alphacoronavirus, Betacoronavirus, Cytomegalovirus, Deltainfluenzavirus, Deltacoronavirus, Gammacoronavirus, Hepacivirus, Hepatovirus, Influenza A virus, Influenza B virus, Influenza C virus, Influenza D virus, Lentivirus, Letovirus, Lymphocryptovirus, Orthopneumovirus, Orthohepadnavirus, Orthopoxvirus, Papillomavirus, Quaranjavirus, Rotavirus, Simplexvirus, and Varicellovirus.

11. The method of claim 7, wherein the viral antigen is derived from a spike protein of Betacoronavirus.

12. The method of claim 7, wherein the fungal antigen is derived from a fungal species that causes a fungal infection selected from the group consisting of aspergillosis, blastomycosis, candidiasis, chromoblastomycosis, cryptococcosis, histoplasmosis, mycetoma, paracoccidioidomycosis, ringworm and tinea versicolor.

13. The method of claim 7, wherein the parasitic antigen is derived from a parasite species that causes a parasitic infection selected from the group consisting of African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, and trichuriasis.

14. The method of claim 5, wherein the non-coding RNA selected from the group consisting of a small interfering RNA (siRNA), a ribosomal RNA (rRNA), a transfer RNA (tRNA), a microRNA (miRNA), and an aptamer.

15. The method of claim 4, wherein the RNA template is a chimeric DNA/RNA oligonucleotide.


SEQUENCE LISTING (9 sequences)

SEQ ID NO: 1 - DNA, 2790 bp, synthetic construct
  source 1..2790: mol_type = other DNA
atgcaatcatttttgaaccgtgtttgcggagtgtccgctgcacgtctcacgccttgtggtacggggacgtctaccgatgtggtgtaccgggcgttcgacatctacaacgataaggtggctggattcgccaagtttctcaaaaccaactgttgtagatttcaggagaaggacgaggatgataacttgatagacagttactttgtggtgaaaagacatacatttagcaattaccagcacgaagaaacgatatataatctgctgaaagattgccctgccgttgctaagcacgacttttttaagtttcgcatcgacggcgacatggtgccccatattagtcgacaacgtctcactaagtacaccatggcagacctggtgtacgccctgaggcactttgacgaggggaactgtgatacattgaaggaaattctcgtcacctacaactgctgtgatgacgactatttcaacaaaaaagattggtatgactttgttgagaacccagatattctccgcgtgtacgccaatctgggggagagagtccgtcaagccttacttaaaacagtccagttctgtgacgcaatgagaaacgcgggaatcgttggcgtgctgacattagataaccaggatcttaatggcaattggtatgacttcggggactttatccaaacaacccctggcagcggcgttcccgtcgtcgattcctactatagtctcttaatgccaattctgactctgactagagccttaaccgccgagagtcatgtcgacacagatttgacaaagccctacataaagtgggatctccttaaatatgattttactgaagaacgactgaagcttttcgaccgctatttcaaatattgggaccaaacttatcaccctaattgtgtgaattgcttagatgatcgctgcattcttcattgcgctaatttcaacgtgttattctcaaccgtgtttcctcctacttccttcggcccattggtgaggaaaatttttgtcgatggtgtcccctttgtggtgtcaacaggctatcactttagggaattgggggtcgtccataaccaggatgtcaatttgcattcctctcggctctccttcaaggagctgctggtgtatgccgcggaccccgctatgcacgccgccagcggaaacctcctcctggacaagaggacaacgtgtttttccgtagcagctcttaccaacaacgttgccttccagacagttaaacccggaaactttaataaagacttctatgattttgctgtttccaaaggcttcttcaaggaggggtcatcagtggagctgaagcatttcttcttcgctcaggacggcaatgcggcgatttctgattatgattattaccgctacaatctgcccacaatgtgtgatatcagacagttattatttgtggtggaagttgtcgacaagtatttcgactgctacgatggaggttgcatcaacgcaaaccaagtgattgttaataacctggacaagagtgcaggcttcccttttaacaagtggggcaaggccagactctactatgactccatgtcttatgaggatcaggacgctctgtttgcgtacacaaagcgcaatgttatcccaacgattacccaaatgaatctgaaatacgctatctctgctaagaatagagctcgtaccgtcgctggagtttcaatctgcagcacaatgactaataggcagttccatcaaaaactgcttaagagcatagcggctacacgtggagcaactgtcgtgattgggactagtaagttctatggcgggtggcacaacatgctcaagacggtatactccgatgtagaaaatccccacctcatgggatgggattaccccaaatgcgatagagcaatgccgaacatgctccggattatggcttctcttgtgcttgcacgaaaacatacaacctgttgtagcctgtcccaccggttctatagactcgctaatgagtgcgcccaggttctcagtgaaatggttatgtgcgggggctctctgtatgtaaaacctggcggcaccagcagtggcgatgccacaactgcctatgcaaactccgtgtttaacatttgccaagccgtgacggcaaacgtgaacgctcttttgtcaaccgacggcaataaaatcgccgacaagtatgtcagaaacctccagcatcggctgtatgagtgtctgtaccgcaaccgggatgtggacactgattttgtgaacgaattctatgcatacttgaggaaacacttcagcatgatgatcctctccgatgacgctgtggtgtgctttaactcaacatatgcatctcaaggactcgttgcgtccattaaaaattttaagtctgttctctattaccagaacaacgtattcatgtccgaagctaaatgctggaccgagactgacttgaccaagggcccacacgaattttgctcccaacataccatgctcgtcaagcaaggggacgattatgtgtatctcccatacccggatccatcacggattttgggagctgggtgctttgtggatgatatcgtgaagaccgatggcaccctgatgatcgaaagattcgtgtcacttgccatcgatgcttatcctttgacgaaacacccaaaccaggaatacgccgacgtcttccatctgtatctgcagtacattcgcaaactgcacgacgaactcacaggccacatgctggacatgtactcagtgatgctcacaaacgataacacatcccggtactgggagcccgagttttacgaagcgatgtacaccccgcatactgtgctgcagtaa

SEQ ID NO: 2 - DNA, 345 bp, synthetic construct
  source 1..345: mol_type = other DNA
atgaataacgagctgagccctgttgcactgaggcagatgagctgcgctgccggtacgacacagacagcttgcaccgatgataatgccttggcctattacaatactactaaaggtggccgtttcgtcctggcgttactgagcgatctgcaggatctcaagtgggctcgttttcccaagtcagacggtactggcaccatttatacagaactggagccaccgtgcagatttgtaacagatacccccaaggggcctaaggttaaatacctgtacttcattaagggactcaataacctgaacagaggcatggtgctcggttccctcgccgctacagtgcggctccagtga

SEQ ID NO: 3 - DNA, 1587 bp, synthetic construct
  source 1..1587: mol_type = other DNA
atggccgagaatgtgaccggattgttcaaggattgtagtaaggtcatcaccggtctccatcctacccaggcaccgacccacttgtctgtcgataccaagtttaagaccgagggcctgtgcgtggacataccaggaattcctaaggatatgacttatcgccgtttgatttccatgatgggcttcaagatgaactaccaggtcaacggttatcctaatatgttcatcacccgcgaggaggccattagacacgtaagagcttggataggattcgatgtagaaggctgtcatgctaccagagaagccgtgggtaccaacctgccgctgcagcttggcttcagcactggcgtgaatctggtggcggtgccgaccggttatgtggatacccctaacaacacagacttctcccgcgtatctgctaaacccccccctggcgaccagtttaagcacctgatccctctcatgtataagggacttccctggaatgtggtgaggatcaaaattgttcaaatgttgagcgacacccttaagaaccttagtgatagggtcgtcttcgtcctctgggctcacggatttgaactcacaagcatgaagtattttgtcaaaattgggccagagcgaacatgttgcctttgcgacagacgtgctacttgcttttctaccgcttccgacacctatgcctgctggcaccactctatcggttttgattacgtatacaatccttttatgatcgacgtgcagcagtggggctttacaggcaatcttcagtcaaaccacgacctttattgccaggtacacggcaatgcccatgtcgcctcctgcgatgccatcatgactcgttgtctcgcagtccacgaatgttttgtgaagagagttgattggacgatcgaatatccaatcattggtgatgagttaaaaataaacgctgcttgccgcaaggtgcagcatatggtggtgaaggcagccctcttagcagataagtttccagtgctgcacgacataggaaaccctaaggccattaagtgtgtcccccaagcggacgtggagtggaaattctacgacgcacagccctgcagtgacaaagcctacaagattgaggagctcttctattcatacgccacacatagtgacaagtttacagatggagtctgtctgttttggaactgcaatgtggacagataccccgctaatagcatcgtttgtcgtttcgacacaagggttctgagcaaccttaatcttccaggctgcgacgggggatccttatacgttaacaaacacgcgtttcacacccctgcctttgataaatccgcttttgtgaacctgaaacaattaccgtttttctactatagtgactctccttgcgagtcccacgggaagcaggtcgtgtcagacatagactacgtgccactgaagtcagctacgtgcataactcggtgcaatttaggaggcgctgtgtgtcggcatcatgctaacgaataccggctgtacctggacgcatacaacatgatgatcagcgccggattttctttgtgggtttacaagcagtttgacacatataacctgtggaatacattcacgagactccagtga

SEQ ID NO: 4 - RNA, 2790 nt, synthetic construct
  source 1..2790: mol_type = other RNA
atgcaatcatttttgaaccgtgtttgcggagtgtccgctgcacgtctcacgccttgtggtacggggacgtctaccgatgtggtgtaccgggcgttcgacatctacaacgataaggtggctggattcgccaagtttctcaaaaccaactgttgtagatttcaggagaaggacgaggatgataacttgatagacagttactttgtggtgaaaagacatacatttagcaattaccagcacgaagaaacgatatataatctgctgaaagattgccctgccgttgctaagcacgacttttttaagtttcgcatcgacggcgacatggtgccccatattagtcgacaacgtctcactaagtacaccatggcagacctggtgtacgccctgaggcactttgacgaggggaactgtgatacattgaaggaaattctcgtcacctacaactgctgtgatgacgactatttcaacaaaaaagattggtatgactttgttgagaacccagatattctccgcgtgtacgccaatctgggggagagagtccgtcaagccttacttaaaacagtccagttctgtgacgcaatgagaaacgcgggaatcgttggcgtgctgacattagataaccaggatcttaatggcaattggtatgacttcggggactttatccaaacaacccctggcagcggcgttcccgtcgtcgattcctactatagtctcttaatgccaattctgactctgactagagccttaaccgccgagagtcatgtcgacacagatttgacaaagccctacataaagtgggatctccttaaatatgattttactgaagaacgactgaagcttttcgaccgctatttcaaatattgggaccaaacttatcaccctaattgtgtgaattgcttagatgatcgctgcattcttcattgcgctaatttcaacgtgttattctcaaccgtgtttcctcctacttccttcggcccattggtgaggaaaatttttgtcgatggtgtcccctttgtggtgtcaacaggctatcactttagggaattgggggtcgtccataaccaggatgtcaatttgcattcctctcggctctccttcaaggagctgctggtgtatgccgcggaccccgctatgcacgccgccagcggaaacctcctcctggacaagaggacaacgtgtttttccgtagcagctcttaccaacaacgttgccttccagacagttaaacccggaaactttaataaagacttctatgattttgctgtttccaaaggcttcttcaaggaggggtcatcagtggagctgaagcatttcttcttcgctcaggacggcaatgcggcgatttctgattatgattattaccgctacaatctgcccacaatgtgtgatatcagacagttattatttgtggtggaagttgtcgacaagtatttcgactgctacgatggaggttgcatcaacgcaaaccaagtgattgttaataacctggacaagagtgcaggcttcccttttaacaagtggggcaaggccagactctactatgactccatgtcttatgaggatcaggacgctctgtttgcgtacacaaagcgcaatgttatcccaacgattacccaaatgaatctgaaatacgctatctctgctaagaatagagctcgtaccgtcgctggagtttcaatctgcagcacaatgactaataggcagttccatcaaaaactgcttaagagcatagcggctacacgtggagcaactgtcgtgattgggactagtaagttctatggcgggtggcacaacatgctcaagacggtatactccgatgtagaaaatccccacctcatgggatgggattaccccaaatgcgatagagcaatgccgaacatgctccggattatggcttctcttgtgcttgcacgaaaacatacaacctgttgtagcctgtcccaccggttctatagactcgctaatgagtgcgcccaggttctcagtgaaatggttatgtgcgggggctctctgtatgtaaaacctggcggcaccagcagtggcgatgccacaactgcctatgcaaactccgtgtttaacatttgccaagccgtgacggcaaacgtgaacgctcttttgtcaaccgacggcaataaaatcgccgacaagtatgtcagaaacctccagcatcggctgtatgagtgtctgtaccgcaaccgggatgtggacactgattttgtgaacgaattctatgcatacttgaggaaacacttcagcatgatgatcctctccgatgacgctgtggtgtgctttaactcaacatatgcatctcaaggactcgttgcgtccattaaaaattttaagtctgttctctattaccagaacaacgtattcatgtccgaagctaaatgctggaccgagactgacttgaccaagggcccacacgaattttgctcccaacataccatgctcgtcaagcaaggggacgattatgtgtatctcccatacccggatccatcacggattttgggagctgggtgctttgtggatgatatcgtgaagaccgatggcaccctgatgatcgaaagattcgtgtcacttgccatcgatgcttatcctttgacgaaacacccaaaccaggaatacgccgacgtcttccatctgtatctgcagtacattcgcaaactgcacgacgaactcacaggccacatgctggacatgtactcagtgatgctcacaaacgataacacatcccggtactgggagcccgagttttacgaagcgatgtacaccccgcatactgtgctgcagtaa

SEQ ID NO: 5 - RNA, 345 nt, synthetic construct
  source 1..345: mol_type = other RNA
atgaataacgagctgagccctgttgcactgaggcagatgagctgcgctgccggtacgacacagacagcttgcaccgatgataatgccttggcctattacaatactactaaaggtggccgtttcgtcctggcgttactgagcgatctgcaggatctcaagtgggctcgttttcccaagtcagacggtactggcaccatttatacagaactggagccaccgtgcagatttgtaacagatacccccaaggggcctaaggttaaatacctgtacttcattaagggactcaataacctgaacagaggcatggtgctcggttccctcgccgctacagtgcggctccagtga

SEQ ID NO: 6 - RNA, 1587 nt, synthetic construct
  source 1..1587: mol_type = other RNA
atggccgagaatgtgaccggattgttcaaggattgtagtaaggtcatcaccggtctccatcctacccaggcaccgacccacttgtctgtcgataccaagtttaagaccgagggcctgtgcgtggacataccaggaattcctaaggatatgacttatcgccgtttgatttccatgatgggcttcaagatgaactaccaggtcaacggttatcctaatatgttcatcacccgcgaggaggccattagacacgtaagagcttggataggattcgatgtagaaggctgtcatgctaccagagaagccgtgggtaccaacctgccgctgcagcttggcttcagcactggcgtgaatctggtggcggtgccgaccggttatgtggatacccctaacaacacagacttctcccgcgtatctgctaaacccccccctggcgaccagtttaagcacctgatccctctcatgtataagggacttccctggaatgtggtgaggatcaaaattgttcaaatgttgagcgacacccttaagaaccttagtgatagggtcgtcttcgtcctctgggctcacggatttgaactcacaagcatgaagtattttgtcaaaattgggccagagcgaacatgttgcctttgcgacagacgtgctacttgcttttctaccgcttccgacacctatgcctgctggcaccactctatcggttttgattacgtatacaatccttttatgatcgacgtgcagcagtggggctttacaggcaatcttcagtcaaaccacgacctttattgccaggtacacggcaatgcccatgtcgcctcctgcgatgccatcatgactcgttgtctcgcagtccacgaatgttttgtgaagagagttgattggacgatcgaatatccaatcattggtgatgagttaaaaataaacgctgcttgccgcaaggtgcagcatatggtggtgaaggcagccctcttagcagataagtttccagtgctgcacgacataggaaaccctaaggccattaagtgtgtcccccaagcggacgtggagtggaaattctacgacgcacagccctgcagtgacaaagcctacaagattgaggagctcttctattcatacgccacacatagtgacaagtttacagatggagtctgtctgttttggaactgcaatgtggacagataccccgctaatagcatcgtttgtcgtttcgacacaagggttctgagcaaccttaatcttccaggctgcgacgggggatccttatacgttaacaaacacgcgtttcacacccctgcctttgataaatccgcttttgtgaacctgaaacaattaccgtttttctactatagtgactctccttgcgagtcccacgggaagcaggtcgtgtcagacatagactacgtgccactgaagtcagctacgtgcataactcggtgcaatttaggaggcgctgtgtgtcggcatcatgctaacgaataccggctgtacctggacgcatacaacatgatgatcagcgccggattttctttgtgggtttacaagcagtttgacacatataacctgtggaatacattcacgagactccagtga

SEQ ID NO: 7 - protein, 929 aa, synthetic construct
MQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 8 - protein, 114 aa, synthetic construct
MNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 9 - protein, 551 aa, synthetic construct
MEGLCVDIPGIPKDMTYRRLISMMGFKMNYQVNGYPNMFITREEAIRHVRAWIGFDVEGEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ